(19) United States
(12) Patent Application Publication
(10) Pub. No.: US 2018/0364225 A1
(43) Pub. Date: Dec. 20, 2018

(54) HIGH THROUGHPUT SCREENING OF SMALL MOLECULES

(71) Applicants: President and Fellows of Harvard College, Cambridge, MA (US); Children's Medical Center Corporation, Boston, MA (US); Dana-Farber Cancer Institute, Inc., Boston, MA (US)

(72) Inventors: Bhavik Nathwani, Boston, MA (US); Darren Yang, Somerville, MA (US); Wesley Philip Wong, Cambridge, MA (US); William M. Shih, Cambridge, MA (US); Andrew Ward, Boston, MA (US)

(73) Assignees: President and Fellows of Harvard College, Cambridge, MA (US); Children's Medical Center Corporation, Boston, MA (US); Dana-Farber Cancer Institute, Inc., Boston, MA (US)

(21) Appl. No.: 16/060,392

(22) PCT Filed: Dec. 7, 2016
(86) PCT No.: PCT/US16/65341

§ 371 Date: Jun. 7, 2018

Related U.S. Application Data
(60) Provisional application No. 62/264,265, filed on Dec. 7, 2015.
(60) Provisional application No. 62/301,699, filed on Mar. 1, 2016.

(51) Int. Cl. G01N 33/552; G01N 33/543

(52) U.S. Cl. CPC G01N 33/552; G01N 33/566; G01N 33/54386; G01N 33/54306

(57) ABSTRACT

Provided herein, in some embodiments, are methods of high-throughput screening of small molecules and related compositions and articles of manufacture.

### RELATED APPLICATION

This application claims the benefit under 35 U.S.C. § 119(e) of U.S. Provisional Application Ser. Nos. 62/264,265 and 62/301,699 respectively filed on Dec. 7, 2015 and Mar. 1, 2016, both entitled “HIGH THROUGHPUT SCREENING OF SMALL MOLECULES”, the entire contents of which are incorporated by reference herein.

### FEDERALLY SPONSORED RESEARCH

This invention was made with U.S. Government support under N00014-15-1-0073 awarded by the U.S. Department of Defense and under DGE1144152 awarded by the National Science Foundation. The Government has certain rights in the invention.

### BACKGROUND

The pharmaceutical industry has benefited from progress in the field of combinatorial chemistry. Preparing large “libraries” of small molecules or peptides, utilizing robotics and fluidics for automation, and large-scale automated image processing routines have enabled high throughput screens, benefiting drug development.

However, despite promise, limitations in the sensitivity of the underlying screening assays limit the identification rate of positive “hits” from such assays. Single molecule force spectroscopies, such as atomic force microscopy (AFM), optical tweezers and magnetic tweezers have traditionally provided a means to study molecular interactions. Such serial approaches are however low throughput and thus of limited use in screening studies.

### SUMMARY

Provided herein is a novel system for interrogating single molecules or single molecular interactions in a highly multiplexed manner. The single molecules or single molecular interactions are interrogated using force. Such force may be applied, for example, via centrifugation of sample holders. In this way, a plurality of single molecules and/or single molecular interactions can be analyzed simultaneously. Moreover, the systems provided herein are uniquely designed so that various conditions can also be applied to different subsets of molecules or molecular interactions, and the effects of such varied conditions on molecules and/or molecular interactions can be assessed simultaneously.

The methods provided herein employ mechanochemistry (i.e., the study of chemical bonds under applied force), and accordingly they are referred to as Multiplexed Mechanochemistry Assays (MMA). In these assays, force such as centrifugal force is applied simultaneously to a plurality of single molecules (or single molecular interactions). This may be accomplished, for example, by coupling a mass-bearing moiety such as a bead to each single molecule being studied. In the case of a molecular interaction, the mass-bearing moiety is coupled to one of the molecules involved in the interaction. Force is applied to the single molecule (or molecular interaction) via the mass-bearing moiety. The force may be varied by varying the rotation rate of the centrifuge, by varying the distance of the single molecule from the center of rotation, and by varying the mass of the mass-bearing moiety. The mass of the mass-bearing moiety may be varied by using moieties of different size (e.g., diameter) and/or by using moieties of different composition. This approach leads to significant improvement in sample throughput particularly compared to state of the art methods such as atomic force microscopy (AFM), optical tweezers and magnetic tweezers.

As well, the improved design of the sample loading system allows the study of a hundred or more different experimental conditions per MMA run. As an example, in some embodiments, up to 320 different experimental conditions are contemplated. This feature also improves throughput by multiple orders of magnitude.

Thus, this disclosure contemplates use of MMA as a high throughput platform to study various types of chemical bonds under mechanical force. The methods may be used to screen molecules based on bond strength and/or dissociation kinetics (e.g., k_{d}). Chemical bonds under analysis may be in a single molecule or they may be bonds between molecules. In either respect, the bonds may be covalent bonds. The large forces available when using high speed ultracentrifugation underscores the ability to interrogate high strength bonds, such as covalent bonds.

Thus, in one aspect, this disclosure provides a method comprising attaching a plurality of single molecules to a surface, wherein each single molecule is conjugated to a bead at its free end; applying a force to each single molecule by moving the bead to which it is conjugated away from the surface, removing the force and locating the bead on the single molecule. Locating the bead may intend, in some instances, determining whether the bead is still attached to the single molecule and/or whether it is no longer attached (referred to herein as “loss of a bead”). In some embodiments, loss of a bead indicates that a bond strength in the single molecule was less than the force applied to the single molecule. In some embodiments, the plurality of single molecules comprise at least a first and a second subset that differ from each other by the size of the bead conjugated to single molecules in the subset.

In some embodiments, the method further comprises subjecting a first portion of the first subset to a first condition and a second portion of the first subset to a second condition.

In some embodiments, the method further comprises subjecting a first portion of the second subset to a first condition and a second portion of the second subset to a second condition.

In another aspect, this disclosure provides a method comprising: attaching a plurality of single molecules to a surface, wherein each single molecule is conjugated to a bead at its free end; applying a force to each single molecule by moving the bead to which it is conjugated away from the surface, removing the force and locating the bead on each single molecule, such as for example determining that the bead is or is not attached to each single molecule. In some embodiments, loss of a bead indicates that a bond strength in the single molecule was less than the force applied to the single molecule. In some embodiments, the plurality of single molecules comprise at least a first and a second subset that are exposed to a first and a second condition respectively during the application of force.

In some embodiments, the method further comprises subjecting a first portion of the second subset to a first force and a second portion of the second subset to a second force, wherein the first and second forces are different from each other.

In some embodiments, the first and second conditions differ from each other in terms of pH, salt concentration, and denaturant content and/or concentration.

In another aspect, this disclosure provides a method comprising: (1) rotating a plurality of surfaces, each surface attached to a plurality of single molecules, each single molecule conjugated to a bead at its free end, for a first time to apply a force to each bead; (2) stopping rotation of the surfaces and counting beads (still) conjugated to each single surface or a portion of a single surface; and (3) repeating steps (1) and (2) at least once. In some embodiments, single molecules attached to a single surface or a region of a single surface experience the same force.

In another aspect, this disclosure provides a method for high throughput screening comprising performing a screening assay on a plurality of molecules under mechanical force, wherein each molecule is under a constant mechanical force, but force experienced by molecules is different.

In another aspect, this disclosure provides a method for high throughput screening comprising performing a screening assay on a plurality of molecules under mechanical force, wherein each molecule is under a constant mechanical force, and wherein a first subset of molecules is exposed to a first condition and a second subset of molecules is exposed to a second condition. In some embodiments, the first condition comprises a library member and the second condition does not comprise the library member.

Various embodiments apply to the various aspects provided herein. These are recited once for brevity.

In some embodiments, the single molecules in the plurality are identical to each other. In some embodiments, the single molecules in the plurality are different from each other.

In some embodiments, the force is a centrifugal force. In some embodiments, the force is in the picoNewton (pN) to nanoNewton (nN) range.

In some embodiments, the surface is a glass or plastic surface.

In some embodiments, the single molecules are attached to the surface using a first affinity binding pair. In some embodiments, the single molecules are conjugated to a bead using a second affinity binding pair, wherein the first and second affinity binding pairs are different from each other.

These and other aspects and embodiments will be described in greater detail herein.

### BRIEF DESCRIPTION OF DRAWINGS

FIG. 1A depicts a front, top perspective view of a first embodiment of a holder.

FIG. 1B depicts a top view of the holder shown in FIG. 1A.

FIG. 2A depicts a front, top perspective view of a second embodiment of a holder.

FIG. 2B depicts a top view of the holder shown in FIG. 2A.

FIG. 3 depicts a plurality of holders stacked on top of each other and held within a carriage.

FIG. 4 depicts a bottom side of the holders of FIGS. 1A and 2A.

FIG. 5 depicts a top-down view into a centrifuge used with the holders of FIGS. 1A and 2A.

FIG. 6 depicts a system that can studied using the methods provided herein. Three micron anti-Dig coated silica microspheres are immobilized on a biotinylated surface using a streptavidin-biotin interaction. The tether is a ˜24 kb segment of lamda (λ) DNA with biotins at one end and digoxigenin (Dig) at the other end.

FIG. 7 depicts the results using different sample loading systems. These results indicate that the methods can be performed using for example a bench-top centrifuge or an ultracentrifuge to apply force, coupled with the different sizes of readily available microspheres. These device/bead combinations result in a wide range of forces, ranging from a few fN to several hundred (˜300) nN, that can be applied to single molecules or single molecular interactions of interest.

FIG. 8 depicts a bar graph showing the relationship between the number of beads still attached to the solid support as a function of time exposed to a certain force. Samples were subjected to ˜42 pN force for 2 minutes before imaging. Four rounds of imaging were conducted. A mono-exponential fit was used to estimate force lifetime. T_{1/2} for Dig-antibody was estimated to be ˜188.49 seconds based on this analysis.

FIG. 9 depicts a fit through force lifetimes measured at different applied forces. This analysis was used to estimate a zero force off-rate of about ˜4 hours, consistent with values found in the literature. The experiment was carried out as described for FIG. 8.

FIG. 10A depicts a front, top perspective view of a third embodiment of a holder.

FIG. 10B depicts a top view of the holder shown in FIG. 10A.

FIG. 11A depicts a front, top perspective view of a fourth embodiment of a holder.

FIG. 11B depicts a top view of the holder shown in FIG. 11A.

FIG. 12A depicts a front, top perspective view of a fifth embodiment of a holder.

FIG. 12B depicts a top view of the holder shown in FIG. 12A.

FIG. 13 depicts a bottom side of the holders of FIGS. 10A, 11A and 12A.

It will be understood that the various elements and features illustrated in the Figures are not drawn to scale.

### DETAILED DESCRIPTION

This disclosure contemplates methods and products for use in screening assays and other analyses of molecular interactions and/or molecular stability under force. These methods involve a novel technique, referred to herein as Multiplexed Mechanochemistry Assay (MMA). The methods allow a range of forces to be applied to a single molecule (or molecular interaction) in a single run. In doing so, it can be used to determine bond strength within a single molecule, including importantly covalent bond strength, or within a molecular interaction. The methods also allow a range of conditions to be applied to a single molecule (or molecular interaction) in a single run. In doing so, the methods may be used to determine conditions that strengthen or weaken a bond within a single molecule, such as a covalent bond, or a molecular interaction.

In an exemplary and illustrative embodiment, the methods may be used to study bond rupture events in a single molecule. Doing so may then lead one of ordinary skill to rank order a number of candidate molecules based on such bond rupture data.

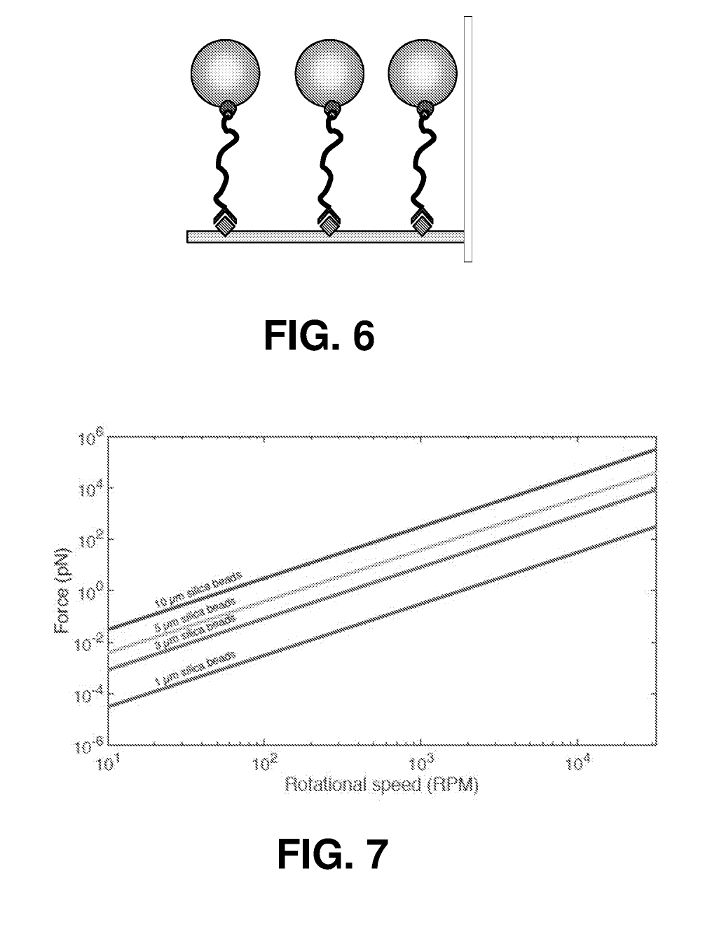

Thus, the disclosure contemplates an assay system in which molecules of interest are attached at one position to a surface, such as a microscope slide, and at another position to a mass-bearing moiety, such as a bead. This configuration is illustrated in FIG. 6 where the molecule of interest is a segment of λ DNA. In this embodiment, the DNA is functionalized at a first position (such as a first end) with streptavidin, rendering it capable of binding to a biotinylated surface at that position. The DNA is functionalized at a second position (such as a second end) with an antigen such as digoxigenin (Dig), thereby rendering this second position capable of binding to an anti-Dig antibody present on the mass-bearing moiety (e.g., the bead). A centrifugal system was designed to apply pre-calibrated forces on a large number of samples (e.g., up to 320 samples) simultaneously. The force that can be applied ranges from a fraction of a pN to ˜30 nN, in some instances. As system validation, we report the results of a study of covalent bond rupture using the methods provided herein.

The system and the methods are referred to as two dimensional multiplexing because they are able to screen a large number of samples and conditions simultaneously. This represents an unprecedented level of “ultraplexing”, where hundreds of experimental conditions can be tested against orders of magnitude variation in force. It provides a dramatic improvement in throughput of individual molecules over conventional single molecule force spectroscopy (SMFS) techniques.

These systems and methods provided herein greatly enhance the ability to probe and understand the relation between force-lifetime and chemistry in single molecular bonds.

It is to be understood that the molecule of interest may be a nucleic acid or it may be a non-nucleic acid including for example a peptide, a protein, a polysaccharide, and a lipid. It may also be a small organic or inorganic molecule of virtually any composition.

The molecule of interest may be a complex comprising two or more subunits. Those subunits may be covalently or non-covalently bound to each other.

The disclosure contemplates a variety of ways in which the molecule of interest may be attached to the surface or the mass-bearing moiety. Examples include biotin-streptavidin, biotin-avidin, ligand-receptor pairs, antigen-antibody pairs, etc. Virtually any affinity pairing may be used provided it remains stable and bound during the analysis. That is, the affinity pair that is used to tether the molecule of interest to the surface or to the bead must not be ruptured prior to the rupture of a bond in the molecule or complex of interest, since if the affinity pair ruptures before the molecule or complex do, then this will lead to a spurious result (e.g., the end user may observe that the bead is no longer present and may conclude, incorrectly, that this is due to a bond rupture in the single molecule as a result of the applied force).

In some embodiments, the molecule of interest is a nucleic acid. In other embodiments, the molecular interaction of interest involves a nucleic acid. In still other embodiments, the molecule or molecular interaction of interest do not include nucleic acids. In these latter embodiments, the molecules being analyzed singly or as part of a molecular interaction may be attached to one or more nucleic acids which in turn is attached to a support or a mass-bearing moiety. In still other embodiments, the molecules are attached directly or indirectly to the support or the mass-bearing moiety.

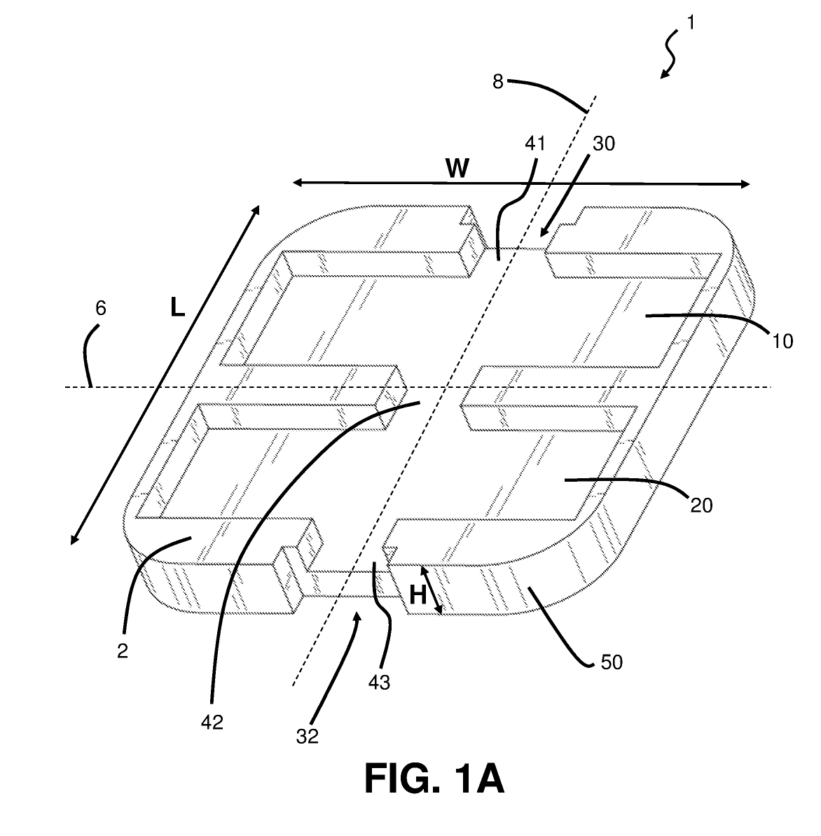

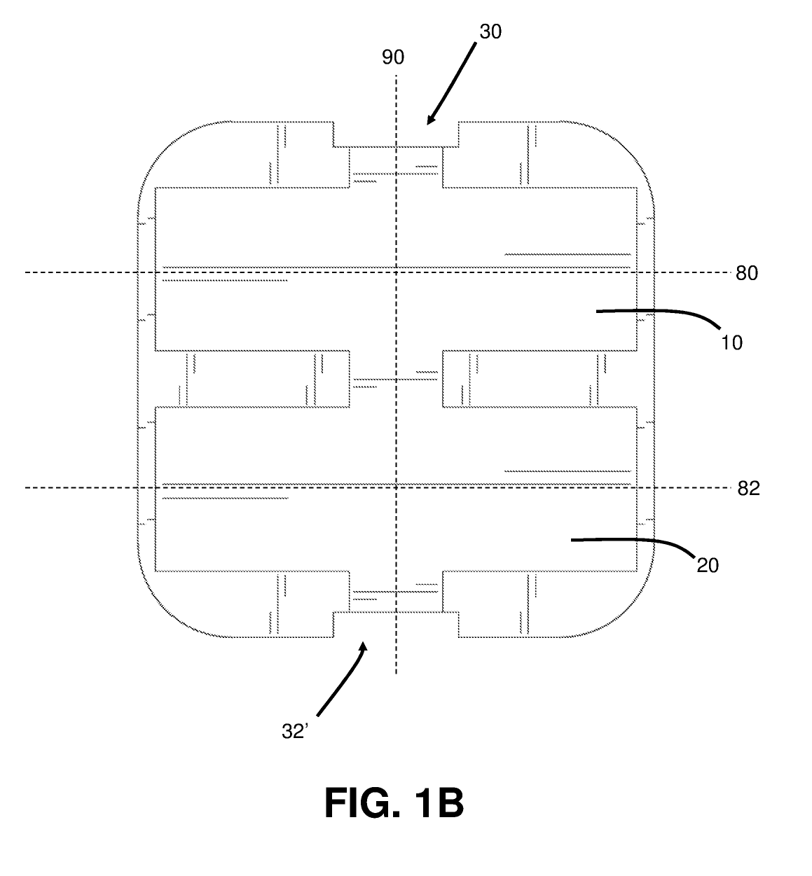

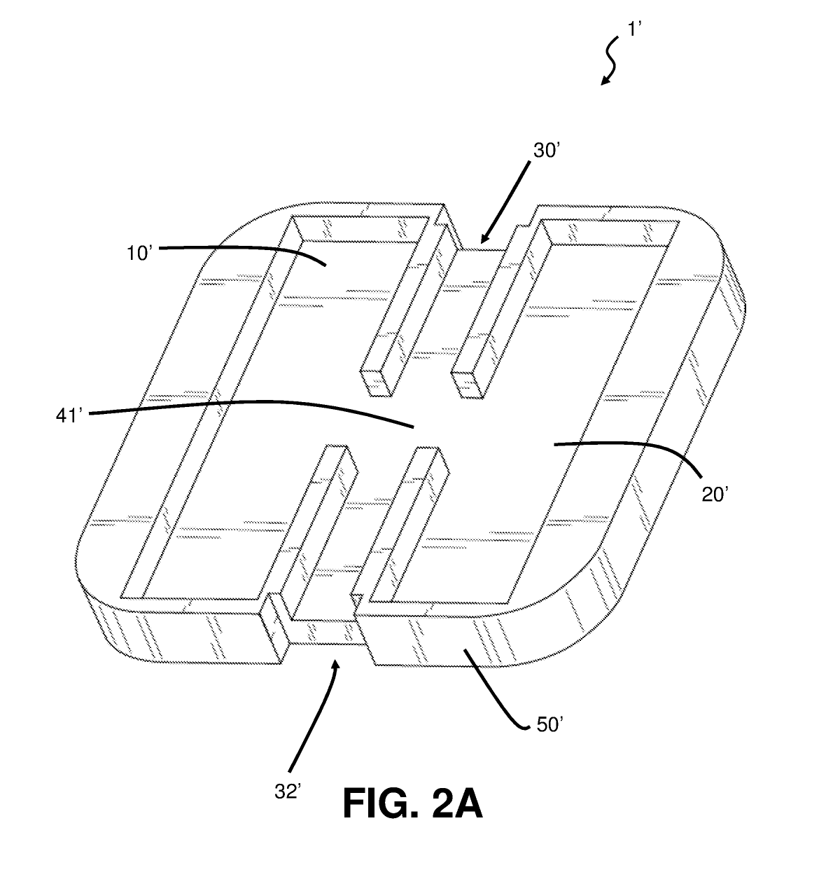

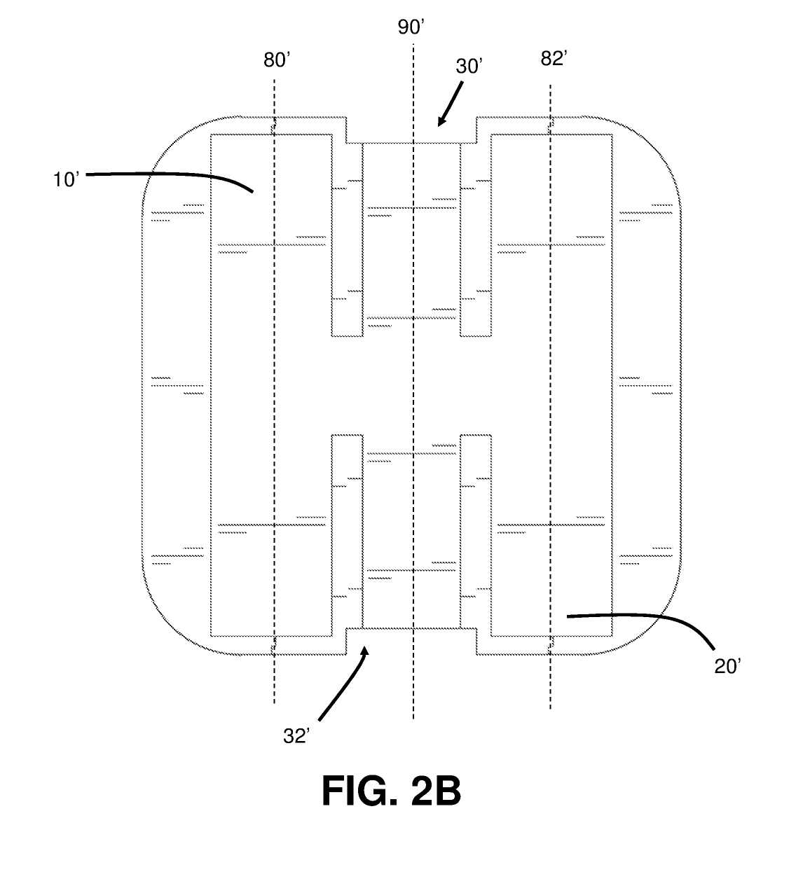

The disclosure contemplates that the molecule of interest may be secured (or tethered) to virtually any support. In some embodiments, the support is one that is capable of being secured and then spun as in a centrifuge such as a bench-top centrifuge or an ultracentrifuge. In some embodiments, such as those illustrated in FIGS. 1A and 2A, the support may be a slide such as a glass or plastic slide, optionally through which the presence and potentially number of beads can be determined.

As will be understood in view of this disclosure, each support has a force, such as a centrifugal force, applied to it (and thus also to the molecule or molecular interaction that is tethered to the support), and then the presence or absence of the mass-bearing moiety is determined. This may be accomplished by removing the support from its holder and imaging or otherwise examining it. The number of visible mass-bearing moieties may be used to indicate the number of bonds that have not yet ruptured as a result of force applied. In some embodiments, the position of the visible mass-bearing moieties may be used to indicate whether a bond has been affected.

The disclosure also contemplates the use of mass-bearing moieties in order to apply force to the molecule or molecular interaction of interest as well as a detectable moiety that is the ultimate visible readout. In other words, in these embodiments, the mass-bearing moiety is used to apply force to the molecule or molecular interaction but it is not the moiety that is ultimately detected or visualized to determine whether a bond has or has not ruptured. This may be accomplished, for example, by conjugating the mass-bearing moiety and a detectable label to the single molecule of interest or to the molecular interaction of interest (e.g., conjugation to one of the molecules involved in the molecular interaction). It is to be understood that the mass-bearing moiety and the detectable label should be positioned in proximity to each other such that once the mass-bearing moiety is detached from the molecule so too is the detectable label. Detectable labels include but are not limited to fluorophores. Alternatively, the mass-bearing moiety may be modified such that it is conjugated to a detectable label. And still another embodiment, the mass-bearing moiety is inherently detectable (e.g., it may be fluorescent or emit a detectable signal).

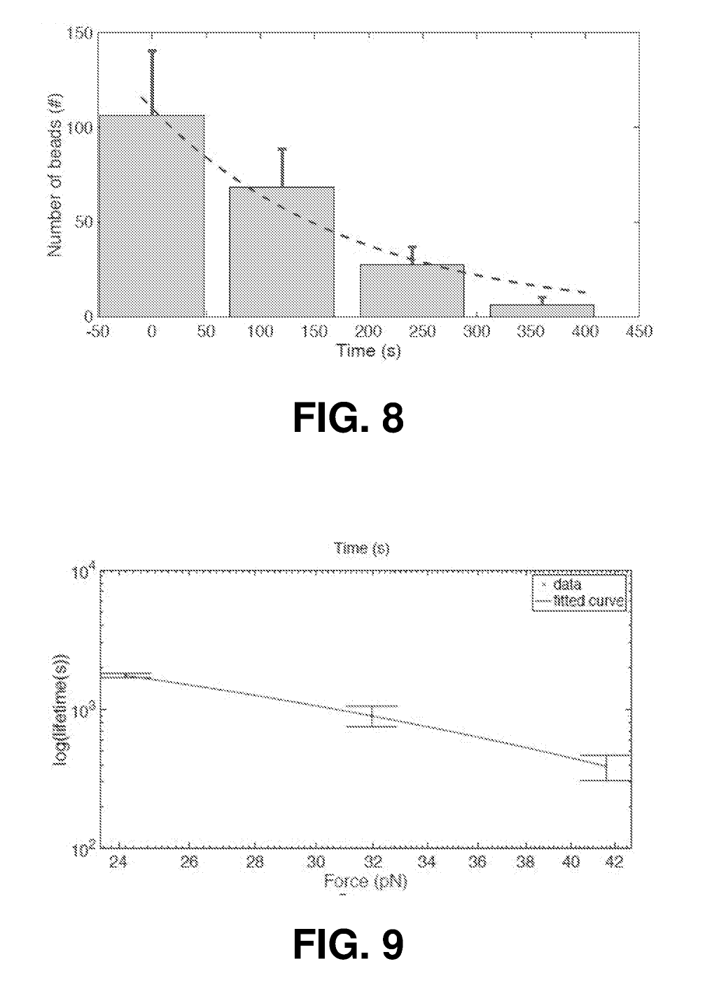

The analysis may be carried out through one or more cycles of force application and imaging, with the number and/or location of mass-bearing moieties determined after each cycle. The time during which force is applied may be the same for all cycles, or it may be different. The results of an embodiment of such an analysis are provided in FIG. 8, where it is clear that the longer the period of force application (e.g., the longer the centrifuge run, or the longer the period of time that a molecule or molecular interaction experiences a force), the fewer mass-bearing moieties (e.g., beads) that remain attached. Such an analysis when performed on different molecules or different molecular interactions may allow an end user to rank order the bond strength between molecules or between molecular interactions. This may be done, for example, by determining the time required to detach 50% of the starting mass-bearing moieties. Other metrics may also be used to compare and contrast bond strengths, molecules, and/or molecular interactions.

The analysis may alternatively or additionally carried out by applying different forces to a molecule of interest or a molecular interaction of interest. This may be accomplished, for example, by conjugating different sized mass-bearing moieties to such molecules (whether single molecules or molecules participating in a molecular interaction). In some embodiments, beads of different sizes (i.e., different diameters) are used to apply different forces on the same molecule. For example, through the use of the holders described herein, two or more supports may be used simultaneously, with one support comprising molecules conjugated to a bead having a diameter d, and another support comprising the same molecules yet conjugated to a bead having a diameter of 2d. The latter beads have a greater mass and thus will apply a larger force to the molecules. Experimental results from such an analysis are shown in FIG. 7, where it is shown that mass-bearing moieties such as silica beads having diameters of 1 micron, 3 microns, 5 microns, and 10 microns, exert increasing forces for a set rotational speed.

Forces applied may be in the range of 1-1000 pN, 1-500 pN, 1-400 pN, 1-300 pN, 1-200 pN, 1-100 pN, 5-100 pN, 5-90 pN, 5-80 pN, 5-70 pN, 5-60 pN, 5-50 pN, 5-40 pN, 5-30 pN, 5-20 pN, 5-10 pN, 10-100 pN, 10-50 pN, 10-40 pN, and 10-30 pN, for example. Bead sizes may vary and may for example in the 1 micron to 10 micron range, including any size in between. Rotational speed that is used to exert force may range in some embodiments from 10¹-10⁶ RPM, 10¹-10⁵ RPM, 10²-10⁴ RPM, 10¹-10³ RPM, 10¹-10² RPM, 10²-10⁶ RPM, 10²-10⁵ RPM, 10²-10⁴ RPM, 10²-10³ RPM, for example. Force may be applied for varied periods of time including but not limited to about 1000 seconds, about 900 seconds, about 800 seconds, about 700 seconds, about 600 seconds, about 500 seconds, about 400 seconds, about 300 seconds, about 200 seconds, about 100 seconds, or about 50 seconds, for example.

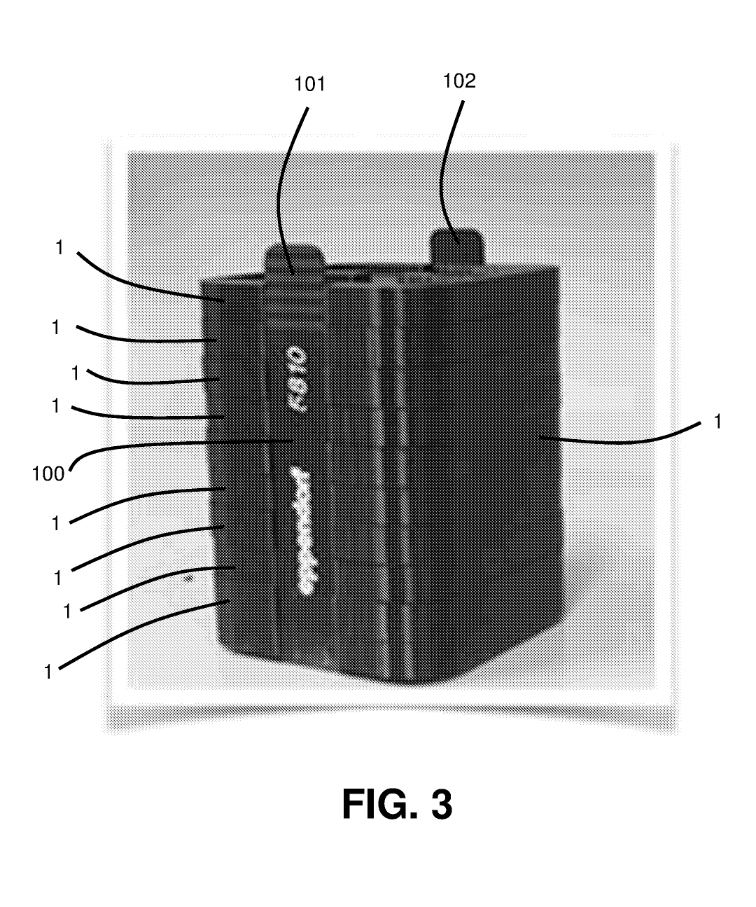

The disclosure further contemplates that the placement of the holder, and thus the sample, in its stack (as illustrated in FIG. 3) will also contribute to a different force being applied to the molecules. For example, a support that is placed in the holder at the bottom of the stack will experience a different force from a support that is placed in the holder at the top of the stack, since the positions of these holders relative to the center of rotation are different.

Half-lives of bonds, molecules and molecular interactions may also be determined using the afore-mentioned analyses. Additionally lifetimes for a molecular interaction may be determined, for example under constant force, and before rupture of a bond using the methods provided herein. Lifetime data can then be used to determine rate constants.

Thus, the methods provided herein, in some embodiments, are directed to an engineered technological solution for the problem of low success rate in small molecule identification during drug screens. The present disclosure provides, in some embodiments, methods for utilizing centrifugal force to modulate the energy landscape of ligand-receptor interactions in a multiplexed framework. The methods provide the following advantages, for example: (1) interrogation of hundreds (e.g., 200 to 500 (e.g., 320), or more) of ligand-receptor interactions during a single experimental run (multiplexing ligand-receptor interactions), (2) a force range from, for example, a few tens of fN to μN, a 9 orders of magnitude spread in force range (force range), and (3) for a given ligand-receptor pair, interrogation across, for example, a 1.72-fold-force range per experimental run (multiplexing force application).

Current reporter-based assays, such as ELISA and luminescence-based reporter assays, are currently used to assay small molecule activity in modulating receptor-ligand interactions. Such reporter-based assays rely on cut-off points, which are determined by comparing the activity of a molecule with a known standard. Typically, molecules with activity higher than cut-off points are considered “hits” and lower than cut-off points are considered “negatives.” Through application of force, the methods provided herein can be used to enhance the “sensitivity” of a given molecular system to small molecule intervention and to find a larger number of hits relative to current assays.

The methods provided herein achieve multiplexing, in part, through running a large number of samples through each centrifugation run. The methods (assays) provide a wide range of available forces, over nine orders of magnitude, for example. Increasing the susceptibility of molecules or molecular interactions such as but not limited to receptor-ligand pairs to exogenous molecules allows identification of hits that would otherwise be missed by conventional assays. Because identification is performed under external force conditions, downstream medicinal chemistry can be utilized to identify derivatives of such hits that would have higher activity.

In some embodiments, the present disclosure provides methods of determining the rupture force of a single molecule or of a single molecular interaction such as but not limited to a ligand-receptor pair or antibody-antigen pair.

In some embodiments, the present disclosure provides methods of determining the rupture force of a single molecule or of a single molecular interaction such as but not limited to a ligand-receptor pair or antibody-antigen pair at different force loading rates.

In some embodiments, the present disclosure provides methods of screening the efficacy of small molecule drug candidates at modulating the bond strength within a single molecule or a single molecular interaction such as but not limited to a ligand-receptor pair or antibody-antigen pair.

In some embodiments, the present disclosure provides methods of screening the efficacy of peptides at modulating the bond strength within a single molecule or a single molecular interaction such as but not limited to a ligand-receptor pair or antibody-antigen pair.

In some embodiments, the present disclosure provides methods of force-based separation and subsequent classification of nucleic acid binding proteins, including classification based on strength of binding. In these embodiments, force may be applied to a molecular interaction between a nucleic acid such as DNA and a nucleic acid binding protein such as a DNA binding protein.

In some embodiments, the present disclosure provides large scale multiplexed methods of determining the binding strength of two or more moieties to each other. Examples include binding strength of transcription factors to nucleic acids or to other factors required and contributing to a transcriptional complex. For example, the analysis may include all transcription factors in a cell such as a mammalian (e.g., human) cell, bacterial cell, fungal cell, or insect cell.

In some embodiments, the present disclosure provides methods of non-specifically adhering molecules, from a bodily fluid sample of a subject (e.g., human subject), to beads. In some embodiments, a surface is decorated with tethers displaying diverse molecules complementary to different types of proteins of pathogenic origin. In some embodiments, the binding energy landscape is used for rapid diagnosis.

Thus, it should be clear that in its broadest sense the disclosure provides methods for characterizing, including comparing and contrasting, bond strength in individual molecules or in molecular interactions that involve two or more molecules. The bonds may be covalent or non-covalent. Bond strength is characterized under force, such as for example centrifugal force. The ability to visual individual mass-bearing moieties, such as beads, also allows the analysis to be conducted on a single molecule basis. In other words, the bond strength of individual molecules or individual interactions can be determined rather than relying on population measurements. Still further, these analyses may be carried out in a rotating device such as a centrifuge, and in doing so may be performed on a plurality of different molecules (or different molecular interactions) and/or may analyze the effect of different conditions on such bond strength (e.g., presence of library member or candidate drug that may weaken or enhance a bond, molecule or molecular interaction). Thus, the method allows an end user to characterize the baseline strength of a bond within a molecule or a molecular interaction and also to characterize the effect of varied conditions on such a bond. The varied conditions may include the presence of one or more candidate modulating agents such as library members or small chemical compounds. Thus, the methods may be used to identify agents that strengthen or weaken bonds of interest, molecules of interest, and/or molecular interactions of interest.

### Holder

According to one aspect, a holder is sized and adapted to hold one or more samples within a centrifuge. For example, samples are coupled to a substrate (or support, as the terms are used interchangeably herein), e.g., a slide, coverslip, cover glass, etc., and the substrate is loaded into the holder. The holder is then inserted into the centrifuge. If the centrifuge has buckets, the holder can be inserted and held within a bucket. If, instead, the centrifuge has open holes other than buckets for insertion of containers, the holder may be sized and shaped to be inserted into those holes.

In one illustrative embodiment, a holder is a plate having a height, which can be also thought of as a thickness. In some embodiments, the height of the holder is its smallest dimension (e.g., as compared to its length and width or diameter).

Within the outer border of the holder, the holder has one or more recesses set into the plate in the height direction of the plate relative to the topmost surface of the holder. The recess is arranged to receive one or more substrates to which sample is coupled. The recess may retain the substrate(s) to the holder via an interference fit, a snap-in engagement, a sliding engagement, a latch, or other suitable retaining arrangement. In some embodiments, the holder has one or more slots into which the substrate is inserted and the opening to the slot is then covered.

The recess for receiving sample substrates may be any suitable shape and size to match a substrate. For example, a recess may be rectangular, square, circular, oval, triangular, pentagonal, octagonal, or any other suitable shape. In some embodiments, multiple substrates can fit within a single recess. E.g., in the case of a rectangular recess, multiple square substrates and/or rectangular substrates that are smaller than the rectangular recess can fit along the length of the rectangular recess. As another example, substrates may be stacked within a recess. E.g., the depth of the recess may accommodate more than one substrate stacked on top of each other.

In some embodiments, the holder may have one or two axes of symmetry, and/or may be rotationally symmetrical. In the case of rotational symmetry, the holder may have rotational symmetry of order 2, 3, 4, 5, 6, infinity (e.g. a circle), or any other suitable order.

Turning to the Figures, one illustrative embodiment of a holder is shown in FIGS. 1A-1B. The holder 1 is a plate having a length L, width W and height H. The height H is smaller than the length L and the width W. The holder has first and second rectangular recesses 10, 20 set into the plate in the height direction H relative to the topmost surface 2 of the holder. The recesses 10, 20 are sized to receive substrates. Each of the recesses 10, 20 can receive a single rectangular substrate that matches the shape and size of the recess. In other embodiments, multiple substrates can be inserted into each recess, e.g. along the length of the recess, and/or stacked within the depth of the recess. The holder 1 has two axes 6, 8 of symmetry and has rotational symmetry of order 2.

The one or more recesses of a holder can be positioned anywhere on the holder—e.g., in the corners, on the sides, in the middle, etc. In the case of a holder having more than one recess, in some embodiments, the recesses may be positioned on the holder in a symmetrical manner to maintain balance.

FIGS. 1A and 2A show two embodiments having different recess placement. In the FIGS. 1A-1B embodiment, recesses 10, 20 have longitudinal axes 80, 82 that run in a direction transverse to a line 90 connecting indents 30, 32. These indents will be discussed in a later section. In the FIGS. 2A-2B embodiment, the recesses 10′, 20′ are rotated 90 degrees relative to the FIG. 1A embodiment, i.e., the longitudinal axes 80′, 82′ of the recesses 10′, 20′ run in a direction parallel to the line 90′ connecting indents 30′, 32′.

Each recess may have one or more corresponding indentations that facilitate removal and/or insertion of a substrate. E.g., the indentation may permit the entry of user's finger to allow the user to more easily grasp and lift and/or insert the substrate. The indentation may appear as an extension of the recess, e.g., the indentation may be adjacent to the recess. In some embodiments, the indentation is at the same recessed height as the recess such that the two are coplanar. In others, the indentation is further indented relative to the recess. An indentation may be on only one side of the recess, or may be on two sides of the recess. E.g., a pair of indentations may flank a recess.

In the illustrative embodiment of FIG. 1A, holder 1 has indentations 41, 42 and 43 that facilitate removal and/or insertion of substrates from recesses 10, 20. Indentations 41 and 42 flank recess 10, and indentations 42, 43 flank recess 20. Indentation 42 is shared by both recesses. The indentations 41, 42 and 43 are at the same recessed height as the recesses 10, 20 such that they all lie on the same plane.

The holder may be shaped and arranged in a stackable design such that multiple holders can be stacked on top of each other. The holder may have flat surfaces on the top and the bottom of the holder such that stacked holders will sit flush with one another. In some embodiments, the top side of the holder has one or more recesses for receiving substrate(s), while the bottom side of the holder is flat.

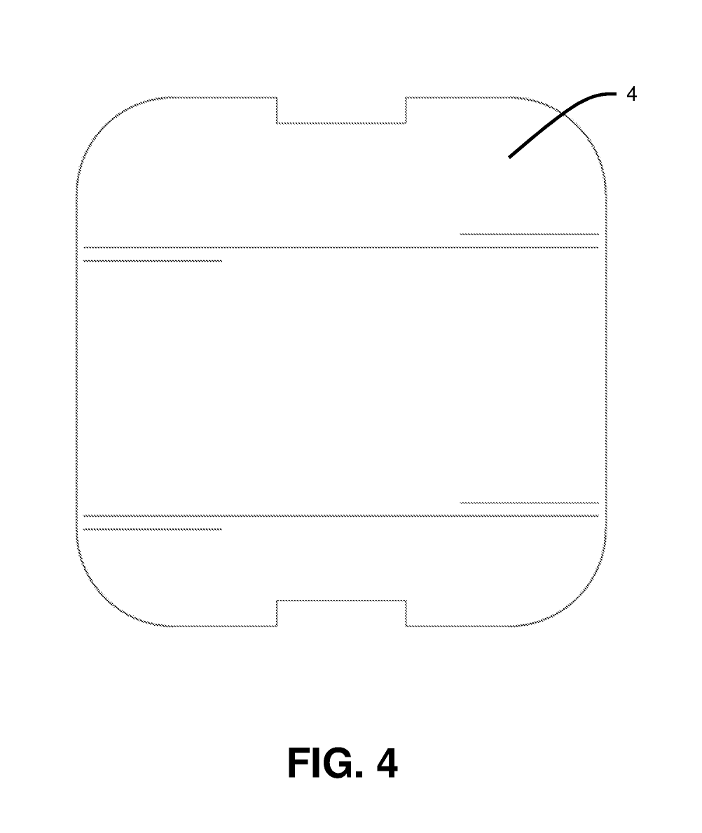

The holders shown in FIGS. 1A and 2A have flat top and bottom surfaces such that the holder is stackable. As seen in FIG. 4, which depicts the bottom side of the holders of FIGS. 1A and 2A, the bottom side 4 is flat. A plurality of holders 1 are shown stacked on top of each other in FIG. 3.

The outer border of the holder may have one or more indents and/or protrusions for compatibility with centrifuge components. For example, some centrifuges have U-shaped carriages that hold stacks of holders together. The holders are stacked within the U-shaped carriage, and the entire assembly is placed into a centrifuge bucket. In such arrangements, the outer border of the holder may have two indents for receiving the arms of the U-shape. In some embodiments, the idents and/or protrusions on the outer border of the holder permit the holder to be compatible with commercially available components for commercially available centrifuges.

In the illustrative embodiment of FIG. 1A, the holder is sized and shaped to fit with a U-shaped carriage. As seen in FIG. 3, a plurality of holders 1 are stacked and top of each other and held within a carriage 100. The carriage may be a commercially available component to be used with a commercially available centrifuge. For example, the holders in FIG. 3 are sized to fit with a U-shaped component 100 for the Eppendorf 5810 centrifuge. As seen in FIG. 1A, the border 50 of holder 1 has two indents 30, 32 to accommodate the arms of the carriage 100. Fitting the arms 101, 102 of the carriage into the holder indents couples the holder to the carriage, preventing the holder from slipping laterally relative to the carriage. The holder may still be able to slide vertically up and down relative to the carriage. In some embodiments, the carriage arms may have detents or other engaging features to prevent vertical sliding between the carriage and the holder.

In some embodiments, the holders may have interlocking features that allow one holder to interlock with another holder. For example, the top of the holder may have a protrusion and the bottom of the holder may have a corresponding indentation. When placing a second holder on top, the protrusion of the top first holder is received into the indentation of the bottom of the second holder.

In an alternative embodiment, the stack of holders may be rotated 90 degrees such that the holders are arranged side-by-side, and then placed inside the centrifuge in that orientation. In other words, instead of placing one holder on top of another holder, holders are arranged side-by-side.

The outer border of the holder may be sized and shaped to fit within a centrifuge bucket or other sample receiving volume in the centrifuge. The outer border of the holder may be square, rectangular, circular, oval, square/rectangular with rounded corners, or any other suitable shape.

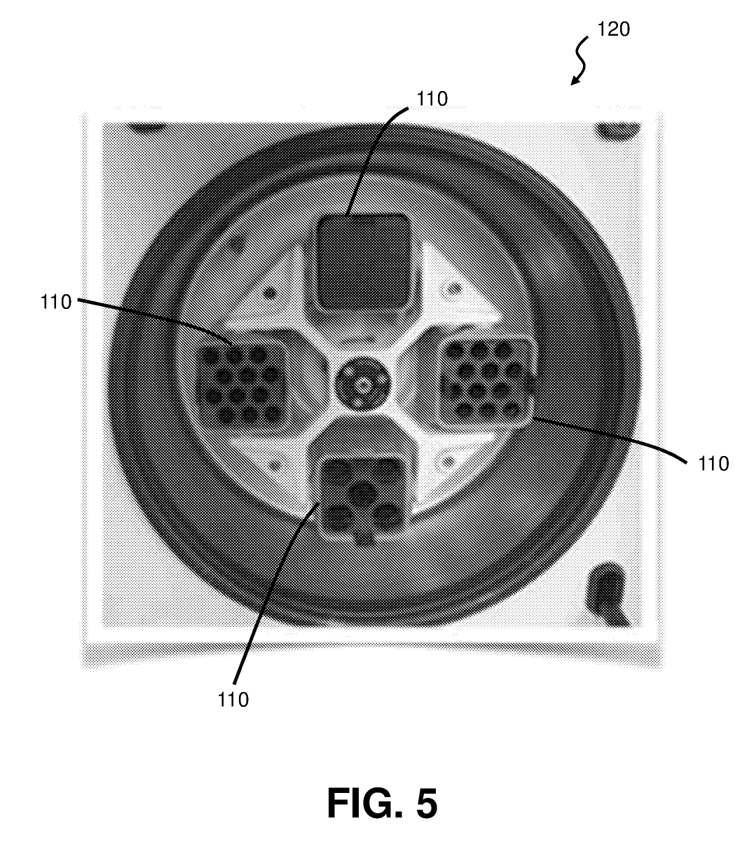

As an example, FIG. 5 depicts a top-down view into a centrifuge 120 to be used with the holders of FIGS. 1A and 2A. The centrifuge buckets 110 are square with rounded corners. Accordingly, as seen in FIGS. 1A and 2A, the borders 50 of the holders are square with rounded corners to match the shape of the centrifuge buckets 110.

The holder may be manufactured via 3D printed, injection molded, die cast, or formed by any other suitable method. The holder may be plastic, thermoplastic, metal, glass, or any other suitable material.

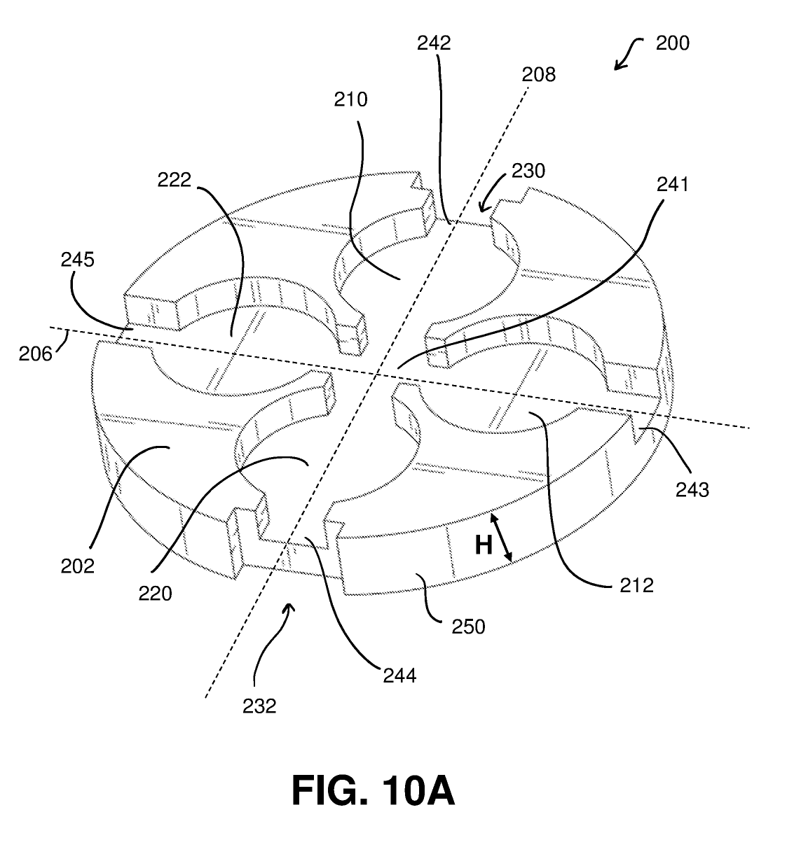

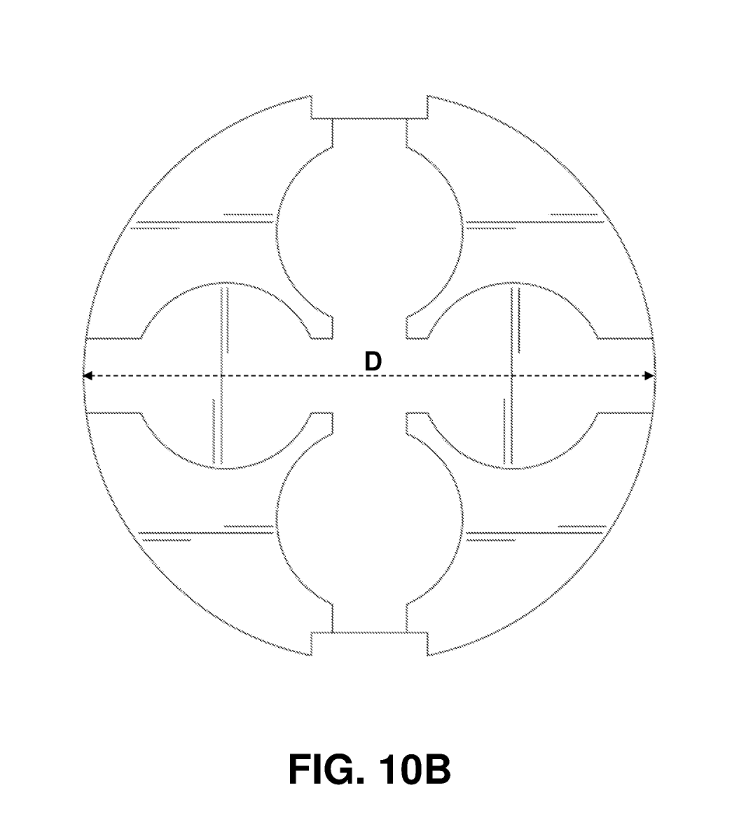

Another illustrative embodiment of a holder is shown in FIGS. 10A-10B. The holder 200 is a circular plate having a height H and diameter D (the diameter may also be referred to as the length). The height H is smaller than the diameter D. The holder has circular recesses 210, 212, 220 and 222 set into the plate in the height direction H relative to the topmost surface 202 of the holder. The recesses 210, 212, 220 and 222 are sized to receive substrates. Each of the recesses 210, 212, 220 and 222 can receive a single circular substrate that matches the shape and size of the recess. In other embodiments, multiple substrates can be inserted into each recess, e.g. stacked within the depth of the recess. The holder 200 has two axes 206, 208 of symmetry and has rotational symmetry of order 2.

The holder 200 has indentations 241, 242, 243, 244 and 245 that facilitate removal and/or insertion of substrates from/into recesses 210, 212, 220 and 222. Indentations 241 and 242 flank recess 210, indentations 241, 243 flank recess 212, indentations 241, 244 flank recess 220 and indentations 241, 245 flank recess 222. Indentation 241 may be shared by all four recesses. The indentations 241, 242, 243 and 244 may be at the same recessed height as the recesses 210, 212, 220 and 222 such that they all lie on the same plane.

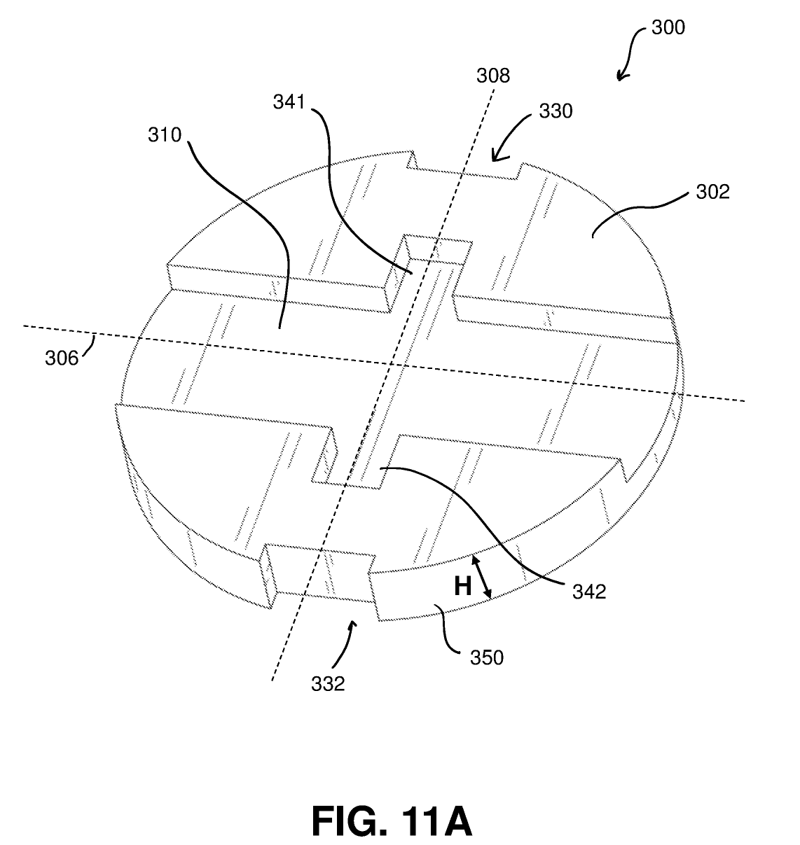

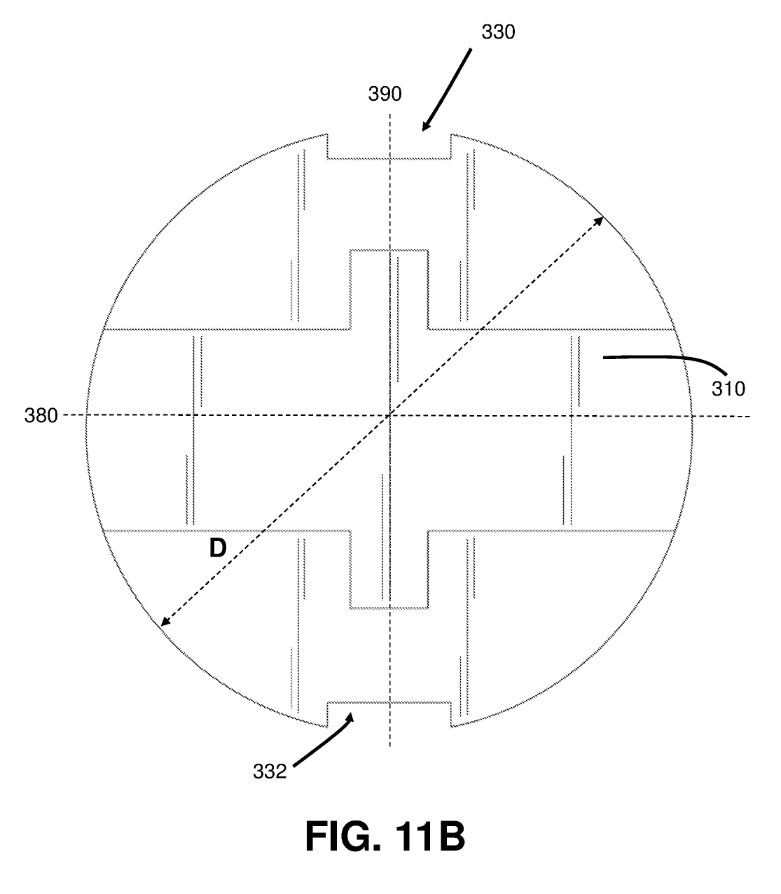

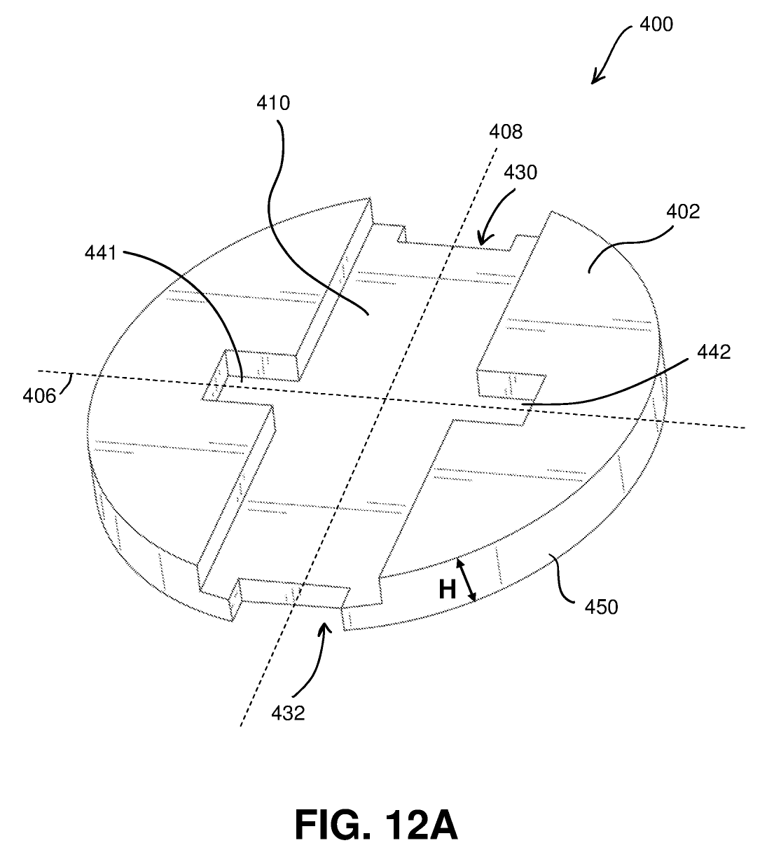

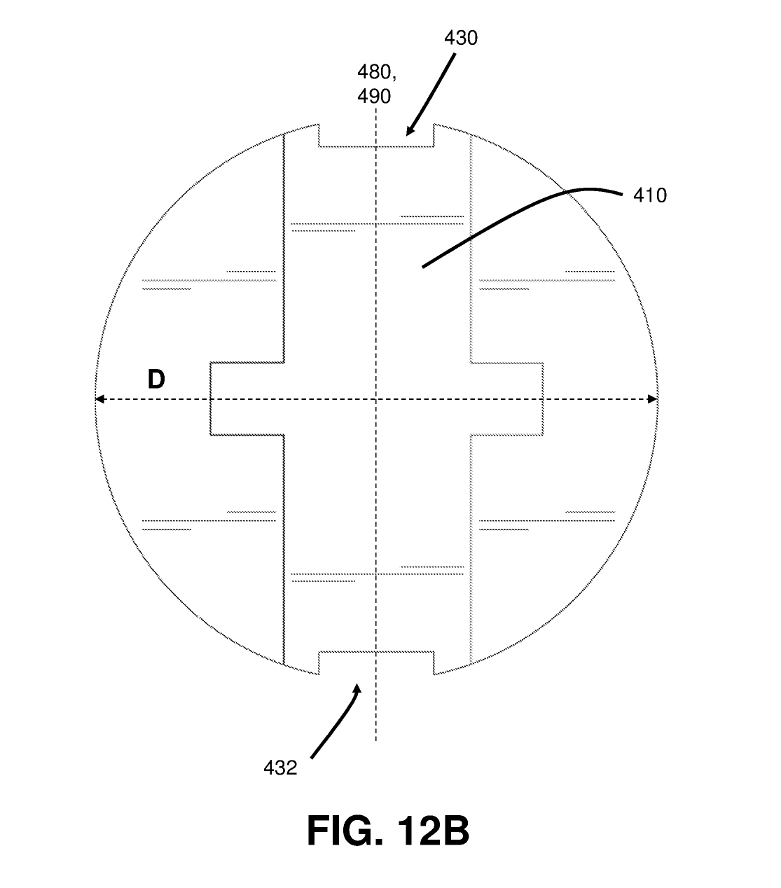

As discussed above, the holder may have any suitably sized and shaped recess for receiving substrates. The embodiments shown in FIGS. 11A and 12A are circular holders with a rectangular recess for receiving one or more substrates. As seen in FIG. 11A, holder 300 has a recess 310 is flanked by two indentations 341, 342 that facilitate removal and/or insertion of substrates from/into the recess 310. Similar to the embodiment of FIG. 10A, the holder 300 has indents 330, 332 that may cooperate with a carriage, and the holder 300 also has a topmost surface 302. The holder 300 has two axes 306, 308 of symmetry and has rotational symmetry of order 2. The holder has a height H and a diameter D, where the height is smaller than the diameter.

The embodiment shown in FIG. 12A is similar to that of FIG. 11A, except the recess is rotated 90 degrees relative to that of FIG. 12A. In the FIGS. 11A-11B embodiment, the recess 310 has a longitudinal axis 380 that runs in a direction transverse to a line 390 connecting indents 330, 332. In the FIGS. 12A-12B embodiment, the recess 410 has a longitudinal axis 480 that runs in a direction parallel to the line 490 connecting indents 430, 432. In some cases, such as with the embodiment of FIG. 12B, the line 490 connecting the indents may be coincident with the longitudinal axis 480 of the recess. Similar to the previous embodiments, holder 400 may also have a topmost surface 402 and indentations 441, 442 that flank the recess 410. The indents 430, 432 of the holder 400 may also cooperate with a carriage. The holder 400 has two axes 406, 408 of symmetry and has rotational symmetry of order 2. The holder has a height H and a diameter D, where the height is smaller than the diameter.

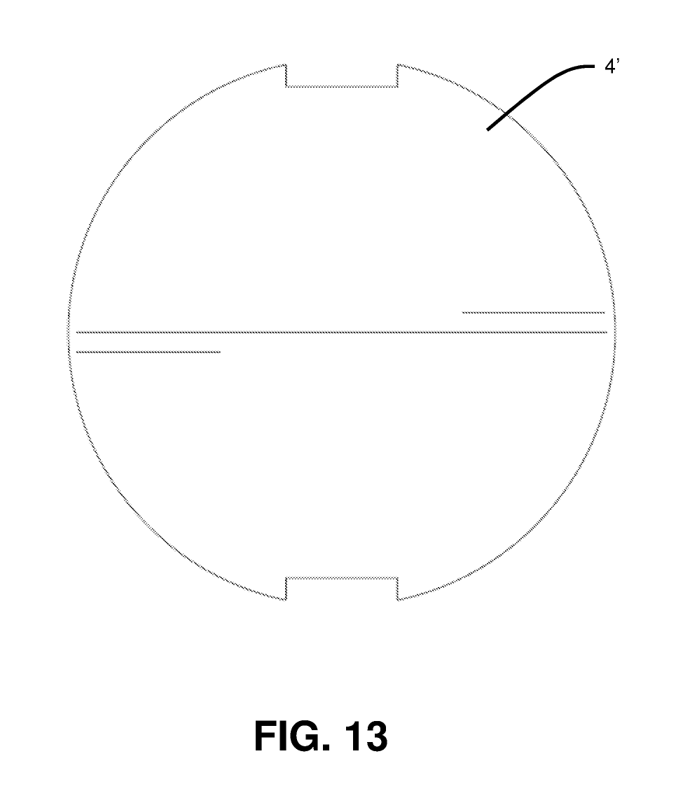

The holders shown in FIGS. 10A, 11A and 12A have flat top and bottom surfaces such that the holder is stackable. As seen in FIG. 13, which depicts the bottom side of the holders of FIGS. 10A, 11A and 12A, the bottom side 4′ is flat. In use with, a plurality of holders may be stacked on top of each other.

The holders of FIGS. 10A, 11A and 12A may be sized and shaped to fit with a U-shape carriage, similar to that shown in FIG. 3. The carriage may be a commercially available component to be used with a commercially available centrifuge. As seen in FIG. 10A, the border 250 of holder 200 has two indents 230, 232 to accommodate the arms of a carriage. As seen in FIG. 11A, the border 350 of holder 300 has two indents 330, 332 to accommodate the arms of a carriage. As seen in FIG. 12A, the border 450 of holder 400 has two indents 430, 432 to accommodate the arms of a carriage.

As discussed above, the outer border of the holder may be sized and shaped to fit within a centrifuge bucket or other sample receiving volume in the centrifuge. In the embodiments of FIGS. 10A, 11A and 12 A, the outer border of the holder is circular and may be sized and shaped to fit within a circular centrifuge bucket or other circular sample receiving container.

### EXAMPLES

A major challenge in high throughput screens is the lack of control over the energy landscapes of molecular interactions. Combined with the limited sensitivity of screening assays, this results in increased false negative (e.g., type II error) rate for screenings. Manipulating the energy landscape of ligand-receptor interaction enables the identification of molecules that would be otherwise disregarded for sub-threshold activity (Tzul, F. O. et al. PNAS 112, E259-E266 (2015)). To improve on the molecular hit rate, centrifugal force is utilized. Briefly, a ligand under study was conjugated to an ssDNA oligo and used as a tether to immobilize silica beads of predetermined size on a cover slip surface. The beads previously were decorated with receptor cognate to the ligand under study. Thus, immobilization of the bead on the tether was mediated by ligand-receptor pairing under study. Concentrations of all reagents were controlled as to ensure that the largest fraction of the beads was immobilized with at most a single tether. When spun at high speeds in a centrifuge, a constant pre-calculated force was applied simultaneously to all beads in a given sample. This is the first mechanism of multiplexing where, unlike conventional approaches, force is applied on all beads on a sample simultaneously. FIGS. 1A-7 outline the design and 3D printed prototype that was used to collect data on the system. FIG. 7 highlights the force range addressable using such designs.

The application of the centrifugal force is the lever that allows modulation of the energy landscape of ligand-receptor interaction. This manifests in progressively decreasing lifetime of the ligand-receptor coupling with application of increasing levels of centrifugal force. By placing samples at different distances from the center of the centrifuge, stacked sample placement, the second mechanism of multiplexing was achieve, namely parallel application of different force levels to a number of samples during a single experimental run. Force mediated lifetime of molecular interactions is estimated by imaging the sample after application of centrifugal force for a short period of time. Repeating this centrifuge/imaging cycle a few times, allows estimation of number of beads “leaving” the tether, for example, rupture of ligand-receptor complex, as a function of time for which the sample was exposed to the set centrifugal force. Fitting a monoexponential, in this prototypical system the interaction between a single ligand-receptor pair is evaluated and therefore this relationship is monoexponential, allowing one to estimate the lifetime of the given molecular interaction under the set level of centrifugal force. Utilizing the second multiplexing mechanism, the same calculation can be performed at various force levels from a single experimental run. Molecular lifetime as a function of applied force allows calculations of various rate constants, K_{off} and Kₒₙ, for the given interaction.

Next, the lifetime for a digoxigenin-antibody pair was measured. Measured lifetime was ˜3 min (177 s) at 42 pN. As FIGS. 8 and 9 highlight, the measured lifetime for this interaction decreased progressively with an increase in applied force. In a high throughput-screening scenario, synergistic action of exogenous compound (enzyme inhibitors or receptor agonists/antagonists) and externally applied force dramatically improves the sensitivity of the ligand-receptor pair to the said compound. This in turn improves the sensitivity of the assay. The inherent multiplexing ability afforded by the platform allows simultaneously screening of effects of a large number of such compounds.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method comprising:
attaching a plurality of single molecules to a surface, wherein each single molecule is conjugated to a bead at its free end;
applying a force to each single molecule by moving the bead to which it is conjugated away from the surface,
removing the force and locating the bead on the single molecule,
wherein loss of a bead indicates that a bond strength in the single molecule was less than the force applied to the single molecule, and
wherein the plurality of single molecules comprise at least a first and a second subset that differ from each other by the force that is applied to them.

2. The method of claim 1, wherein the single molecules in the plurality are identical to each other.

3. The method of claim 1, wherein the single molecules in the plurality are different from each other.

4. The method of any one of claims 1-3, wherein the force is a centrifugal force.

5. The method of any one of claims 1-4, wherein the surface is a glass or plastic surface.

6. The method of any one of claims 1-5, wherein the single molecules are attached to the surface using a first affinity binding pair.

7. The method of any one of claims 1-6, wherein the single molecules are conjugated to a bead using a second affinity binding pair, wherein the first and second affinity binding pairs are different from each other.

8. The method of any one of claims 1-7, further comprising subjecting a first portion of the first subset to a first condition and a second portion of the first subset to a second condition.

9. The method of any one of claims 1-8, further comprising subjecting a first portion of the second subset to a first condition and a second portion of the second subset to a second condition.

10. The method of any one of claims 1-9, wherein the force is in the picoNewton (pN) to nanoNewton (nN) range.

11. The method of any one of claims 1-10, wherein the first and second subsets differ from each other by the size of the bead conjugated to single molecules in each subset.

12. The method of any one of claims 1-10, wherein force is centrifugal force and the first and second subsets differ from each other by their distance from the center of rotation.

13. A method comprising:
attaching a plurality of single molecules to a surface, wherein each single molecule is conjugated to a bead at its free end;
applying a force to each single molecule by moving the bead to which it is conjugated away from the surface,
removing the force and locating the bead on each single molecule,
wherein loss of a bead indicates that a bond strength in the single molecule was less than the force applied to the single molecule, and
wherein the plurality of single molecules comprise at least a first and a second subset that are exposed to a first and a second condition respectively during the application of force.

14. The method of claim 13, wherein the single molecules in the plurality are identical to each other.

15. The method of claim 13, wherein the single molecules in the plurality are different from each other.

16. The method of any one of claims 13-15, wherein the force is a centrifugal force.

17. The method of any one of claims 13-16, wherein the surface is a glass or plastic surface.

18. The method of any one of claims 13-17, wherein the single molecules are attached to the surface using a first affinity binding pair.

19. The method of any one of claims 13-18, wherein the single molecules are conjugated to a bead using a second affinity binding pair, wherein the first and second affinity binding pairs are different from each other.

20. The method of any one of claims 13-19, further comprising subjecting a first portion of the first subset to a first force and a second portion of the first subset to a second force, wherein the first and second forces are different from each other.

21. The method of any one of claims 13-20, further comprising subjecting a first portion of the second subset to a first force and a second portion of the second subset to a second force, wherein the first and second forces are different from each other.

22. The method of any one of claims 13-21, wherein the force is in the picoNewton (pN) to nanoNewton (nN) range.

23. The method of any one of claims 13-21, wherein the first and second conditions differ from each other in terms of pH, salt concentration, denaturant content and/or concentration, presence or absence of a putative bond-strength modulating agent.

24. A method comprising:
(1) rotating a plurality of surfaces, each surface attached to a plurality of single molecules, each single molecule conjugated to a bead at its free end, for a first time to apply a force to each bead;
(2) stopping rotation of the surfaces and counting beads conjugated to each single surface or a portion of a single surface; and
(3) repeating steps (1) and (2) at least once,
wherein single molecules attached to a single surface or a region of a single surface experience the same force.

25. A method for high throughput screening comprising
performing a screening assay on a plurality of molecules under mechanical force, wherein each molecule is under a constant mechanical force, but force experienced by molecules is different.

26. A method for high throughput screening comprising
performing a screening assay on a plurality of molecules under mechanical force, wherein each molecule is under a constant mechanical force, and wherein a first subset of molecules is exposed to a first condition and a second subset of molecules is exposed to a second condition.

27. The method of claim 26, wherein the first condition comprises a library member and the second condition does not comprise the library member.

28. A holder comprising:
a top surface and a bottom;
a first recess set into the holder in a height direction relative to the top surface of the holder such that the first recess and the top surface are on different planes, the first recess having a depth to receive one or more substrates to which sample is coupled; and
first and second indents at opposing sides of the plate, the first and second indents adapted to cooperate with a carriage.

29. The holder of claim 28, wherein the first recess is rectangular.

30. The holder of claim 28, wherein the first recess is circular.

31. The holder of any one of claims 28-30, wherein an outer border of the holder is square.

32. The holder of any one of claims 28-30, wherein the outer border has rounded corners.

33. The holder of any one of claims 28-30, wherein an outer border of the holder is circular.

34. The holder of any one of claims 28-33, further comprising a first indentation adjacent the first recess.

35. The holder of claim 34, wherein a height of the first indentation is the same as a height of the first recess such that the first indentation and the first recess are coplanar.

36. The holder of claim 34, further comprising a second indentation, wherein the first and second indentations flank the first recess.

37. The holder of any one of claims 28-36, wherein the first and second indents are both rectangular.

38. The holder of any one of claims 28-37, wherein the holder has rotational symmetry of order 2.

39. The holder of any one of claims 28-38, wherein a height of the holder is less than a length of the holder.

40. The holder of any one of claims 28-38, wherein the height of the holder is less than a width of the holder.

41. The holder of any one of claims 28-40, wherein a longitudinal axis of the first recess runs in a direction transverse to a line connecting the first and second indents.

42. The holder of any one of claims 28-40, wherein a longitudinal axis of the first recess runs in a direction parallel to a line connecting the first and second indents.

43. The holder of any one of claims 28-42, further comprising a second recess set into the holder in the height direction relative to the top surface of the holder such that the second recess and the top surface are on different planes.

44. The holder of claim 43, wherein the first and second recesses are coplanar.
